(19) United States
(12) Patent Application Publication
(10) Pub. No.: US 2008/0236249 A1
(43) Pub. Date: Oct. 2, 2008

(54) Method for identification of persons based on the analysis of volatile substances

(76) Inventor: Gonzalo Fernandez de la Mora, Madrid (ES)

Correspondence Address: Juan Fernandez de la Mora, 80 Cold Spring Street, New Haven, CT, 06511, US

(21) Appl. No.: 12/008,186

(22) Filed: Jan. 9, 2008

Related U.S. Application Data
(60) Provisional application No. 60/879,426, filed on Jan. 9, 2007.

(51) Int. Cl. G01N 33/88

(52) U.S. Cl. 73/23.35; 73/23.37; 73/31.02; 250/282

(57) ABSTRACT

The breath, body fluids or skin secretions of persons contain numerous volatile substances at concentrations permitting their chemical analysis directly from surrounding ambient gas. Based on the different odor patterns revealed by such analyses for different individuals, a non-contact method is taught that permits the identification of persons and is relatively immune to evasion.

### CROSS REFERENCE TO RELATED APPLICATION

This application claims the benefit of priority to U.S. Provisional Patent Application 60/879,426, filed on Jan. 9, 2007.

### REFERENCES

[1] Juan Fernández de la Mora and Pablo Martinez-Lozano, Method for detecting volatile species of high molecular weight, US provisional patent application, submitted Apr. 4, 2006, turned into and non-provisional U.S. patent application Ser. No. 11/732,770, submitted on Apr. 4, 2007. A related publication has appeared in [2]
[2] P. Martinez-Lozano and J. Fernández de la Mora, Electrospray ionization of volatiles in breath, Int. J. Mass Spectrometry, 265 (1): 68-72, 2007
[3] Dustin J. Penn, Elisabeth Oberzaucher, Karl Grammer, Gottfried Fischer, Helena A. Soini, Donald Wiesler, Milos V. Novotny, Sarah J. Dixon, Yun Xu and Richard G. Brereton, Individual and gender fingerprints in human body odour, J. Royal. Soc. Interface (2007) 4, 331-340
[4] Sarah J. Dixon, Yun Xu, Richard G. Brereton, Helena A. Soini b, Milos V. Novotny, Elisabeth Oberzaucher, Karl Grammer, Dustin J. Penn, Pattern recognition of gas chromatography mass spectrometry of human volatiles in sweat to distinguish the sex of subjects and determine potential discriminatory marker peaks, Chemometrics and Intelligent Laboratory Systems 87 (2007) 161-172

### FIELD AND BACKGROUND OF THE INVENTION

This invention relates to the problem of identifying persons. Many effective methods to do so have been developed, among which fingerprints have played a key role. Alternative methods such as visual or digital comparison of the person's face with a photograph, use of alphanumerical codes or signatures, examination of physical features of the person, such as height, iris structure, speech, etc., have been practiced. Each of these methods has advantages and complications. Fingerprints are sufficiently complex to be highly specific, yet, are simple enough to be amenable to fast recognition algorithms. Techniques have developed to scan fingerprints electronically, without the inconvenience of traditional ink marking. Currently, the US uses fingerprint control at its borders for non-US citizens. However, fingerprinting requires direct contact, and is often found degrading due in part to its association to criminal investigations. As a result, its routine implementation has found resistance, as evident from the fact that, when it is used, it is used only with foreign visitors, and only once at border controls. Voice recognition algorithms are non-contact, and have had a considerable technical development. Voice recognition has a strong potential for identity confirmation with willing individuals. But it is also open to recognition-evasion maneuvers. For instance, a concrete criminal being searched within a large number of persons based on voice recognition can easily deform his speech. In cases of personality theft, properly trained persons have also shown a remarkable ability to imitate other people's voices, while untrained persons can used recorded speech. Humans have an impressive capacity to recognize a large number of previously known faces by brief visual examination, but are less accurate when the identification process is based on comparison of a photograph with an unknown face. Electronic face recognition algorithms have so far been even more prone to errors than humans. Furthermore, many methods are available to confuse both types of recognition process. Recognition algorithms based on observation of the subject's iris are fairly effective, but because of their requirement of an imaging system in offensive proximity with a delicate organ, this method tends to be even less acceptable than fingerprint-based recognition. As a result, the few existing digital recognition techniques that are truly effective are too invasive for use under most usual circumstances, while other more acceptable approaches are inadequately accurate, or subject to evasion. There is hence a need for a recognition method that combines the requirements of acceptable invasiveness for routine utilization and high recognition accuracy, and whose pattern cannot be manipulated by the subject. The purpose of this invention is to provide one such method.

Uses of the invention. The potential applications of acceptable and accurate person recognition or identification methods are numerous, and only a few will be named here. An obvious one is the elimination of use of authentic documentation by persons other than their legal owner at airport controls or other identity control sites such as national borders, in money exchange operations, at restricted meetings, etc. These and other similar situations are sufficiently common for fingerprinting to be conventionally unaccepted, yet exemplify circumstances where highly insecure conditions can be created by identity theft.

Prior breath-analysis art. The starting point for the present invention is a recent US Provisional patent application by Martinez-Lozano and Fernandez de la Mora [1] (published following this application as [2]), showing that mixing human breath with a spray of charged particles leads to the formation of numerous ions characteristic of the breathing individual, at concentrations such that they can be readily detected and quantified by existing analytical instruments. Examples of such instrumentation include atmospheric pressure ionization mass spectrometers (API MS), and differential mobility analyzers (DMAs). Numerous studies had previously shown that the human breath contains a rich mixture of species, including several hundred different chemicals positively identified. What is new in [1] is that many of the vapors reported there have negligible background levels in ambient air, and many have molecular masses between 200 and 600 Dalton. In contrast, prior breath analysis techniques based on more conventional gas chromatographic (GC) and mass spectrometric (MS) analyses had been restricted to high volatility vapors having ambient concentrations comparable to those observed in breath, and whose masses were in all cases near or below 200 amu. The aforementioned invention [1] emphasizes the usefulness of such breath spectra in the identification of sicknesses, for instance, for early cancer diagnosis. No systematic recognition of the chemical nature of the mass peaks identified is reported in [1], although later related progress was later included in [2]. A positive link between any observed breath vapors and metabolites known to be specific markers of concrete cancers is also still missing. However, reference [1] argues convincingly that there must be a causal relation between a health problem and a corresponding anomalous breath spectrum. This reasoning rests on the well established fact that trained dogs are capable of identifying patients of certain cancers with impressive accuracy by simply smelling them, or by smelling samples from their breath. Particularly noteworthy is the fact that this olfactive cancer diagnosis takes place at an early stage, when no known analytical instrument or protocol is able to detect any anomaly. It is therefore apparent that highly specific volatile species closely linked to cancers must exist and must be the basis of the dog's recognition process. This reasonable hypothesis is further strengthened by the quantitative determination of Martinez Lozano and Fernandez de la Mora [1] of the sensitivity level of their API-MS technique, which is in the range of 10⁻¹² atmospheres, and is comparable to the detection thresholds generally attributed to dogs.

Breath analysis for identity determination. With this background, we proceed now to argue the reasons why breath analysis has singular potential not taught in reference [1] to solve the person-identification problem under consideration. Note first that the dog's nostrils can smell not only cancers. Dogs are also known to recognize persons through doors or other barriers that preclude visual identification. This recognition is widely believed to be based on olfaction, evidently implying that the odors associated to different individuals are sufficiently characteristic to make them unambiguously distinguishable from all other individuals. Hence the same reasoning used in [1] to conclude that API mass spectra of breath should permit accurate medical diagnoses, leads to the new realization that the mass spectrometric detection of odors coming from breath, skin, or other human sources should provide an accurate means for recognizing individuals. This inference, based on the evidence available from dogs, is in fact powerfully confirmed by the mass spectra shown in [1] (reproduced here as FIGS. 1 and 2) for two different individuals, revealing the presence of several mass peaks specifically associated to one of the individuals and not with the other, and of many peaks common to both individuals but present in them at quite different concentrations.

The potential of mass spectrometry or other sophisticated chemical analytical instrumentation for sickness recognition has been noted previously in other settings. This includes a large scientific literature in the fields of proteomics and metabolomics for medical diagnosis, based mainly on bodily liquid (blood, urine, tears, etc.) analysis, but including also a substantial breath analysis literature. Person recognition based on body odor has also been proposed previously [3] (also more recently in [4]), based on chemical analysis of sweat, rather than breath. These studies indicate an ability to make certain distinctions, for instance of the sex of the person. Although the complex protocols used so far for sweat analysis make the approach far less practical than the method here proposed based on breath, these studies provide evidence of the pattern differences existing between various individuals, and of the potential to exploit these differences for recognition purposes. Note also that the present invention is not restricted to breath analysis. It is based on the direct analysis of volatiles associated to a person, irrespective of whether such volatiles originate from breath, sweat, or other cutaneous sources of volatiles. This direct analysis can be done in real time. In contrast, the sweat sampling technique proposed in references [3-4] requires collection of condensed material, and subsequent relatively time consuming analysis.

Pattern manipulation. Unlike his voice, an individual has relatively little control over his odors. This is evident from the fact that, either bathed or sweating, a dog is capable of smelling its master. Furthermore, note that air in the lung comes essentially into equilibrium with all volatile substances dissolved in the blood. Breath therefore contains a faithful record of blood substances, in addition to other possible vapor sources from the mouth. By ingesting a special diet or holding some products on the mouth or nose, one may add a number of characteristic species to one's blood and breath. But one cannot so easily suppress the hundreds of other volatile species present in the bloodstream as a result of ordinary metabolic processes. Consequently, intelligent selection of invariant vital species for pattern recognition has a high potential to be immune to evasion maneuvers. Furthermore, due to the high information density of a mass spectrum, the addition of special smelly species to one's breath cannot interfere with the detection of most other species naturally present. Hence, such odor manipulations are ineffective, and could also be easily recognized and exploited as an additional warning sign.

Pattern recognition of odor spectra. A mass spectrum can be simply characterized as a finite set of pairs of values (m/z, I)ᵢ, i=1, 2, etc., where m/z represents the ratio mass/charge for each of the ions identified in the spectrum, and I is its corresponding signal amplitude. For ions whose amplitude exceeds a certain threshold, the ratio m/z can be determined with considerable accuracy, going from three to more than six significant figures depending on the resolution of the mass spectrometer used. A key component of the pattern associated to a mass spectrum is contained in the series of masses. This series is readily digitized and compared to a stored digital standard. It is sufficiently complex and different from the background to be immediately recognized from other odors. It is to a first approximation almost identical from one person to the next (FIG. 2), giving a certain universality to the human odor pattern. Although some of these characteristic peaks will appear or disappear depending on the circumstances (fasting vs. feasting; diet, sickness, etc.), many among them are unaffected by such features, and can form the basis for a solid pattern recognition process. Indeed, FIG. 1 shows the presence of a very large number of peaks present in one individual and completely absent from the background. The amount of information stored in this series of hundreds of mass peaks, each containing up to six digits, is indeed phenomenal. Although the masses of all chemical species are derived from a finite number of isotopes of a finite number of elements, the possible combinations of atoms is vast, even when restricting these combinations only to the most common organic species containing H, N, O, C, and S, and even when restricting the analysis to vapors with molecular weight below 1,200 amu.

In conclusion, this invention teaches a method for recognizing persons based on the odors they produce. The method compares a discrete set of characteristics previously found to be highly representative of the odor of one person, with those later found on another or the same person. These characteristics may include the mass or electrical mobility and the relative intensity of selected vapors, but is not restricted to only these. Nor is this invention restricted to recognition schemes relying on API-MS analyses of breath, but includes also other schemes to analyze the component vapors emitted by persons, not only through their breath, but also from other parts of their bodies. The invention includes also more invasive protocols that may not be generally acceptable for routine identification, but would be appropriate under special situations. Within the later class we include vapors produced by spit, urine, faeces, sweat and other body fluids or solids, as well as oily or fatty components produced by pores in the skin, ears, hair, etc. In cases where the analysis is based on prior ionization of odor vapors, the invention is not restricted either to the particular ionization method used in [1], but includes also alternative techniques such as photo-ionization or electrosprays of non-polar species, that would be more appropriate to detect non-polar substances such as oils or fats.

The new method can be implemented without offensive contact, and is relatively immune to manipulation by the individual.

### BRIEF DESCRIPTION OF THE DRAWINGS

FIG. 1 compares a mass spectrum obtained by ionizing volatile substances carried in the breath of one individual with a mass spectrum similarly obtained by ionizing the backgound gas, showing susbstantial differences between both

FIG. 2 compares mass spectra obtained by ionizing volatile substances carried in the breath of two different individuals, showing a high agreement between the characteristic peaks from both individuals. However, the peak intensity (height) patterns, and even a few special peaks are quite different for both individuals.

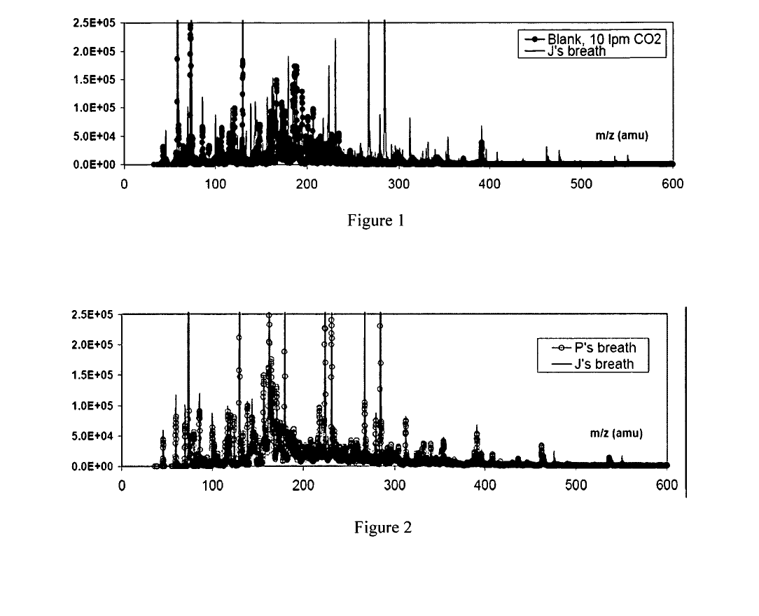

### A MORE DETAILED DESCRIPTION OF THE INVENTION

In one embodiment of the invention, the subject whose identity one wishes to verify by odor analysis is asked to exhale in the vicinity of a sampling point, aiming his breath towards the inlet of a tube through which a certain flow rate q of ambient gas is continuously sampled. This gas may be drawn first into a dehumidifying device to avoid interference between water vapor in the breath and the analytical method used. Subsequently, the sampled vapor is introduced in a charger, where some vapor molecules are ionized and subsequently introduced into an API-MS for mass analysis. The preferred vapor charger is identical to that described in detail in [1], and will not be further detailed here. For reference, the spectrum of the background atmospheric gas is also determined immediately before and after the sample breath is analyzed. In another embodiment of the invention, the MS is replaced by another analytical instrument such an ion mobility spectrometer or a differential mobility analyzer. In still another embodiment, the charger is not based on an electrospray, but on a different type of ionizing method, such as a radioactive source, a source of photons, etc.

What is claimed:

1. A method to identify an unknown person based on comparing a set of characteristics of the volatile substances produced by said unknown person with a previously selected set of said characteristics of said volatile substances produced by a known person, where said volatile substances are sampled directly from the ambient gas surrounding said unknown person and said known person.

2. A method according to claim 1 where said comparison makes use also of the characteristics of volatile substances present in the ambient.

3. A method according to claim 2 where said set of characteristics are derived from ions produced by ionizing said volatile substances with a suitable ionization scheme.

4. A method according to claim 3 where said set of characteristics are part of the mass spectrum of said ions

5. A method according to claim 3 where said set of characteristics are part of the mobility spectrum of said ions

6. A method according to claim 3 where said suitable ionization scheme includes an electrospray source

7. A method according to claim 3 where said suitable ionization scheme includes a source of ionizing radiation.

8. An apparatus to identify persons including the following essential components:
a) means to sample volatile substances directly from a region of the atmosphere, before an unknown person approaches the vicinity of said region, and while said person is in the vicinity of said region
b) means to determine a set of characteristics associated to said sampled volatile substances
c) means to compare said set of characteristics associated to said sampled volatile substances with certain reference standards, with the aim of determining or confirming the identity of said unknown person.
